(19) United States
(12) Patent Application Publication
(10) Pub. No.: US 2026/0207634 A1
(43) Pub. Date: Jul. 23, 2026

(54) NOVEL FORMULATIONS COMPRISING CANNABIS

(71) Applicant: NUTRAMAX LABORATORIES, INC., Lancaster, SC (US)

(72) Inventor: David Griffin, Lancaster, SC (US)

(21) Appl. No.: 19/568,130

(22) Filed: Mar. 16, 2026

Related U.S. Application Data
(62) Division of application No. 17/793,817, filed on Jul. 19, 2022.
(60) Provisional application No. 62/963,228, filed on Jan. 20, 2020.
(60) Provisional application No. 62/968,213, filed on Jan. 31, 2020.
(60) Provisional application No. 63/024,570, filed on May 14, 2020.

(51) Int. Cl. A61K 31/00; A61K 9/00; A61K 36/185

(52) U.S. Cl. CPC A61K 31/658; A61K 9/0019; A61K 9/0031; A61K 9/0056; A61K 36/3482

(57) ABSTRACT

Cannabis-containing compositions comprising at least one cannabinoid, provided in a gel formulation are provided. The compositions may include cannabidiol. The source of the cannabidioid may be cannabis, a cannabis extract, or an industrial hemp oil extract. Methods of treating and/or reducing symptoms of an inflammatory response and/or inflammation are described, comprising administering to a subject in need thereof the disclosed compositions.

This U.S. divisional patent application claims priority to U.S. application Ser. No. 17/793,817, filed Jul. 19, 2022, which is a national stage application of International Application No. PCT/US2021/014162, filed Jan. 20, 2021, which claims priority to U.S. Provisional Patent Application Ser. Nos. 62/963,228 filed on Jan. 20, 2020, 62/968,213 filed on Jan. 31, 2020, and 63/024,570 filed on May 14, 2020, the entirety of the contents of each of which is incorporated herein by reference.

### TECHNICAL FIELD

The present application relates to formulations comprising cannabis, and related methods of use. In one embodiment, the formulations comprise cannabis derived from an industrial hemp plant for administration to a subject.

### BACKGROUND

Cannabis is a genus of flowering plants in the family Cannabaceae. Plants in this family contain compounds known as cannabinoids, such as Δ⁹-tetrahydrocannabinol (THC), cannabidiol (CBD), cannabichromene (CBC), cannabigerol (CBG), and cannabidivarin (CBDV). While THC is known for its psychoactive characteristics, other cannabinoids such as CBD and CBC are not psychoactive. It should be appreciated that the word “cannabinoids” is used in this disclosure to mean any compound that interacts with a cannabinoid receptor and other cannabinoid mimetics.

Cannabinoids have been shown to, among other things, help ameliorate pain and various inflammatory conditions. Cannabinoids, such as THC and CBD, have been found to decrease the production and release of proinflammatory cytokines, including interleukin-1β (IL-1B), interleukin-6 (IL-6), interferon β, from lipopolysaccharide (LPS) activated cells. CBD, in particular, accomplishes this in a number of ways, including, but not limited to: reducing the activity of the NF-κB pathway (a primary pathway regulating the expression of proinflammatory genes); upregulating the activation of the STAT3 transcription factor (an element of homeostatic mechanisms inducing anti-inflammatory events); decreasing levels of mRNA for the Socs3 gene (a main negative regulator of STATs and particularly of STAT3); and decreasing activation of the LPS-induced STAT1 transcription factor (a key player in interferon β-dependent proinflammatory processes).

Information regarding appropriate dosing, optimal formulations, or long-term effects of cannabis in mammals and avians is at present limited. In addition, these compounds are relatively costly; for these reasons, the ability to provide optimal formulations to potentially minimize dosages and still achieve beneficial effects is desirable for both safe and cost-effective administration, while achieving relative or greater efficacy. The present disclosure relates to effects of different preparations comprising cannabis extracted from industrial or other hemp plants, typically formulated for oral, topical, or other administration routes.

### SUMMARY

The present disclosure relates to formulations comprising cannabis, and related methods of use. In accordance with the purposes and benefits described herein, one contemplated embodiment of the present disclosure relates to cannabis-containing compositions comprising at least one cannabinoid, provided in a gel formulation. The compositions may be formulated in unit dosage forms suitable for one or more of oral, rectal, intravenous, subcutaneous, intramuscular, transdermal, transmucosal, and topical administration. In embodiments, the at least one cannabinoid comprises a natural or a synthetic cannabidiol. In embodiments, the source of the at least one cannabinoid is cannabis, a cannabis extract, an industrial hemp oil extract, or combinations.

In embodiments, the compositions may further comprise terpenes and/or flavonoids. In embodiments, the compositions may further comprise excipients and/or carriers selected from the group consisting of vegetable oil, fish oil, krill oil, maltodextrin, calcium carbonate, dicalcium phosphate, tricalcium phosphate, microcrystalline cellulose, dextrose, rice flour, magnesium stearate, stearic acid, croscarmellose sodium, sodium starch glycolate, crospovidone, sucrose, vegetable lactose, gums, methylcellulose, povidone, carboxymethylcellulose, corn starch, and mixtures thereof. In embodiments, the compositions may further comprise one or more additional active ingredients selected from the group consisting of analgesics, non-steroidal anti-inflammatory drug compounds (NSAID), disease-modifying antirheumatic drugs (DMARDs), corticosteroids, anakinra (an interleukin-1 receptor antagonist), COX-2 inhibition, gamma-aminobutyric acid-B (GABAB) receptor agonists, such as baclofen, GABAA potentiating drugs, such as the benzodiazepines, tumor necrosis factor (TNF)-inhibiting drugs, glucosamine, glucosamine salt, chondroitin, chondroitin salt, hyaluronic acid, Boswellia serrata extract, acetyl-11-keto-boswellic acid, methylsulfonylmethane, collagen type II, L-ergothionine, resveratrol, nutritional supplements, and combinations thereof.

In embodiments, the compositions may be formulated for oral administration to an avian and/or a mammalian subject. The mammalian subject may be selected from the group consisting of a human, dog, cat, horse, camel, or cow. In embodiments the mammalian subject is a cat.

In another aspect, the disclosure describes methods for treating and/or reducing symptoms of an inflammatory response and/or inflammation, comprising administering to a subject in need thereof cannabis-containing compositions comprising at least one cannabinoid, the composition being provided in a gel formulation. As described above the at least one cannabinoid may comprise a natural or synthetic cannabidiol. The cannabidiol source may be cannabis, a cannabis extract, an industrial hemp extract, or combinations.

In embodiments the disclosed methods include formulating the compositions as described above to provide unit dosage forms suitable for various administration routes as described above. In embodiments, the compositions may be formulated for oral administration to an avian and/or a mammalian subject. The mammalian subject may be selected from the group consisting of a human, dog, cat, horse, camel, or cow. In embodiments the mammalian subject is a cat.

In embodiments the disclosed methods include formulating the compositions as described above to further comprise terpenes and/or flavonoids. In embodiments the disclosed methods include formulating the compositions as described above to further comprise excipients and/or carriers selected from the group consisting of vegetable oil, fish oil, krill oil, maltodextrin, calcium carbonate, dicalcium phosphate, tricalcium phosphate, microcrystalline cellulose, dextrose, rice flour, magnesium stearate, stearic acid, croscarmellose sodium, sodium starch glycolate, crospovidone, sucrose, vegetable gums, lactose, methylcellulose, povidone, carboxymethylcellulose, corn starch, and mixtures thereof. In embodiments the disclosed methods include formulating the compositions as described above to further comprise one or more additional active ingredients selected from the group consisting of analgesics, non-steroidal anti-inflammatory drug compounds (NSAID), disease-modifying antirheumatic drugs (DMARDs), corticosteroids, anakinra (an interleukin-1 receptor antagonist), COX-2 inhibition, gamma-aminobutyric acid-B (GABAB) receptor agonists, such as baclofen, GABAA potentiating drugs, such as the benzodiazepines, tumor necrosis factor (TNF)-inhibiting drugs, glucosamine, glucosamine salt, chondroitin, chondroitin salt, hyaluronic acid, Boswellia serrata extract, acetyl-11-keto-boswellic acid, methylsulfonylmethane, collagen type II, L-ergothionine, resveratrol, nutritional supplements, and combinations thereof.

### BRIEF DESCRIPTION OF THE DRAWINGS

The accompanying drawing figures incorporated herein and forming a part of the specification, illustrate several aspects of preparations comprising cannabis according to the present disclosure, and together with the description serve to explain certain principles thereof.

FIG. 1 illustrates a mean time-concentration curve of plasma CBD levels for an oil formulation compared to a gel formulation according to the present disclosure;

FIG. 2 shows individual animal plasma CBD levels for the oil formulation of the disclosure;

FIG. 3 shows individual animal plasma CBD levels for the gel formulation of the disclosure;

FIG. 4 illustrates a mean time-concentration curve of plasma THC levels for the oil formulation compared to the gel formulation according to the present disclosure; and

FIG. 5 shows individual animal plasma THC levels for the oil formulation of the disclosure; and

FIG. 6 shows individual animal plasma THC levels for the gel formulation of the disclosure.

Reference will now be made in detail to the present preferred embodiments of the disclosed cannabis-containing formulations; examples of these are illustrated in the accompanying drawing figures.

### DETAILED DESCRIPTION

According to certain contemplated embodiments, the present disclosure relates to novel formulations comprising cannabis for administration to any subject. In embodiments, the subject may be a mammal, an avian, a reptile, or other. In embodiments, the disclosure relates to novel formulations comprising cannabis for administration to mammals, including without intending any limitation humans, canines, equines, and felines.

According to certain contemplated embodiments, the present disclosure relates to novel formulations comprising cannabis for administration to a subject, which formulations may be provided for oral, topical, or other methods of administration. Administration of the cannabis-comprising formulations in various dosage forms is contemplated. For example, the composition may comprise a unit dosage form, including but not limited to pharmaceutical dosage forms suitable for oral, rectal, intravenous, subcutaneous, intramuscular, transdermal, transmucosal, and topical administration.

Examples of dosage forms include, but are not limited to a tablet, a capsule, a powder that can be dispersed in a liquid or sprinkled on food, a liquid such as a solution, suspension, or emulsion, a soft gel/chew capsule, a chewable bar, a gel, a paste, or other convenient dosage form known in the art. In some embodiments, the composition comprises an orally administrable dosage form such as a tablet, a capsule, a paste, a gel, a soft chewable treat, or combinations. The dosage forms may be formulated for immediate release, extended release or delayed release. The composition may be coated or uncoated.

In one contemplated embodiment, the present disclosure relates to novel formulations comprising cannabis provided in a gel or paste formulation for oral administration. In another embodiment, the present disclosure relates to novel formulations comprising cannabis extract provided in a gel formulation for oral administration. In yet another contemplated embodiment, the present disclosure relates to novel formulations comprising cannabis oil extract provided in a gel formulation for oral administration. In still other contemplated embodiments, the present disclosure relates to novel formulations comprising cannabis oil extract comprising CBD provided in a gel formulation for oral administration. In still other contemplated embodiments, the present disclosure relates to novel formulations comprising CBD alone provided in a gel formulation for oral administration.

Cannabis as used in this disclosure can include any component of plants in the Cannabaceae family, such as, for example, the leaves, fruits, seeds, flowers, roots, and stalk of the plants, along with any extracts, isolates, synthetics, powders, oils, or other derivatives thereof and therefrom. The disclosure includes, moreover, compositions which contain mixtures or combinations of cannabis plant components along with any extracts, isolates, synthetics, powders, oils, or respective derivatives thereof and therefrom. For example, the cannabis leaves, fruits, seeds, roots, flowers, and/or stalks can be air dried, freeze dried, drum dried, spray dried, heat dried and/or partial vacuum dried in a hygienic area and then ground into a powder, including in a cold commercial grinding process. This cold commercial process protects the plant's antioxidant, analgesic, and anti-inflammatory properties from oxygen, light, and heat by cryogenically milling the plant materials.

Extracts from the leaf, fruit, seed, root, flower, and/or stalk can be prepared using any conventional extraction methods, including any suitable solvent and/or temperature regime. The cannabis plant components can also be harvested from the plant when they are mature and the oil can be removed through conventional practices, including commercial squeezing or extraction methods that avoid heat, light and oxygen to prevent damaging the vitamins, minerals, antioxidants and other active ingredients found in the plant solids, including cold press extraction methods. The present disclosure can also utilize the cake of the cannabis species, which is the byproduct of the process of pressing in order to extract the oil. This process keeps the vitamins, minerals and other active ingredients chemically undamaged during processing. The cannabis plant components as described above can then be further processed through mixing. Cannabis plant components and combinations and mixtures thereof described above are all intended to be included within the present disclosure. Thus, in some embodiments, the composition and/or method of the present disclosure comprises a use of one cannabis plant component. In some embodiments, the composition and/or method of the present disclosure comprises a use of one or more cannabis plant components. In some embodiments, the composition and/or method of the present disclosure comprises a use of a powder, an extract, an oil, or a seed cake, or a mixture or combination thereof.

Cannabis oil extract as used in this disclosure can include any and all oil extracts from any plant in the family Cannabaceae, or any mixtures or combinations thereof. Such oil extract may be further combined with any components, extracts, isolates, powders, oils, synthetics, or other derivatives from cannabis plants and further contemplates any mixtures or compounds or combinations thereof. In one contemplated embodiment, the cannabis oil extract comprises CBD, and may be extracted from non-psychoactive hemp (also known as industrial hemp), which contains a THC concentration of no more than 0.3% on a dry-weight basis. The disclosure contemplates, however, cannabis oil extract comprising CBD from any cannabis plant, and may, for example, include greater than 0.3% THC on a dry-weight basis or be extracted from psychoactive marijuana or any plant in the family Cannabaceae. In yet another contemplated embodiment, a cannabis oil extract comprising CBD further comprises a full spectrum of all compound classes obtained from the cannabis plants. In yet another contemplated embodiment, a cannabis oil extract comprising CBD further comprises terpenes and flavonoids. In some embodiments of the present disclosure, the oil is a hemp oil and may be a commercially available preparation, such as NMXCB1220™ from Travco Products, Inc., Lancaster, SC.

In still other contemplated embodiments, CBD may be sourced from any plant in the family Cannabaceae or synthesized from other sources. In still other contemplated embodiments, pure or substantially pure CBD may be administered. CBD (CAS No. 13956-29-1) has a molecular formula of C₂₁H₃₀O₂ and the IUPAC nomenclature of 2-[(1R,6R)-3-methyl-6-prop-1-en-2-ylcyclohex-2-en-1-yl]-5-pentylbenzene-1,3-diol, and may be sourced from a number of commercial suppliers. The compounds and combinations thereof described above are all intended to be included within the present disclosure.

In embodiments, the cannabis compositions may be contained in acceptable excipients and/or carriers for appropriate formulation. The carrier may be a liquid, paste, gel, gelcap, capsule, powder, solid tablet (coated or non-coated), or the like. Suitable excipients and/or carriers include vegetable oil, fish oil, krill oil, maltodextrin, calcium carbonate, dicalcium phosphate, tricalcium phosphate, microcrystalline cellulose, dextrose, rice flour, magnesium stearate, stearic acid, croscarmellose sodium, sodium starch glycolate, crospovidone, sucrose, vegetable gums, lactose, methylcellulose, povidone, carboxymethylcellulose, corn starch, and the like (including mixtures thereof). The various ingredients and the excipient and/or carrier may be mixed and formed into the desired form using conventional techniques.

In one aspect, the present disclosure provides methods of preventing and/or reducing an inflammatory response and/or inflammation in a subject. In embodiments, the subject is a human. In embodiments, the subject is a canine. In embodiments, the subject is an equine. In embodiments, the subject is a feline. In one aspect, the present disclosure provides methods for managing inflammatory disorders or generally reducing inflammatory burden of a human or non-human animal. Accordingly, in one embodiment the present disclosure provides a method of preventing and/or reducing an inflammatory response and/or inflammation in one or more tissues, the method including directly or indirectly delivering the compositions of the present disclosure to the one or more tissues of the subject.

In another aspect, the present disclosure provides methods for managing joint health or joint disease/disorder (e.g., arthritis) in a subject. In embodiments, the subject is a human. In embodiments, the subject is a canine. In embodiments, the subject is an equine. In embodiments, the subject is a feline. In certain embodiments, the compositions of this disclosure can be used in methods for treating, supporting, maintaining, or managing joint cartilage, minimizing cartilage degradation, promoting healthy joints by protecting cartilage integrity, diminishing the action of enzymes that affect joint health, improving joint movement and/or function, alleviating joint pain, alleviating joint stiffness, improving joint range of motion and/or flexibility, promoting mobility, and/or any combination thereof. Accordingly, in one embodiment the present disclosure provides a method for treating, supporting, maintaining, or managing joint cartilage, minimizing cartilage degradation, promoting healthy joints by protecting cartilage integrity, diminishing the action of enzymes that affect joint health, improving joint movement and/or function, alleviating joint pain, alleviating joint stiffness, improving joint range of motion and/or flexibility, promoting mobility, and/or any combination thereof. The method includes directly or indirectly delivering the compositions of the present disclosure to the joint cartilage of the subject.

The skilled artisan will appreciate that the novel formulations of the present disclosure can be administered in any amount, including but not limited to a physiologically effective or acceptable dosage. As used herein, “a physiologically effective dosage” may include an amount which is administered under any defined dosing regimen for either clinical, pharmaceutical, medicinal, veterinary, dietary or nutritional purposes. Thus a “physiologically effective dosage” or a “physiologically acceptable dosage” may include a therapeutically effective dosage, a pharmaceutically acceptable dosage, a veterinary acceptable dosage, a nutraceutically acceptable dosage, a dietary acceptable dosage and a nutritionally acceptable dosage, all of which are included for use in the present disclosure.

The formulations of the present disclosure have several advantages over conventional therapies for connective tissue disorders in human and non-human subjects, such as excellent safety profiles. This is in part related to the fact that these compounds occur normally in various foods. Another characteristic shared by the compounds is tendency for a slow onset of action. Pharmaceuticals, such as NSAIDs, tend to cause sudden changes in the symptoms of disease. The compounds described herein work more slowly, by normalizing structures and functions within the body.

Some embodiments of the present disclosure may relate to formulations and therapeutic administration of the formulations in combination with one or more active ingredients that are commonly prescribed or used for treatment of and/or prophylaxis of a joint or inflammatory disease/disorder in a subject including, but not limited to, analgesics, non-steroidal anti-inflammatory drug compounds (NSAID), disease-modifying antirheumatic drugs (DMARDs), corticosteroids, anakinra (an interleukin-1 receptor antagonist), COX-2 inhibition, gamma-aminobutyric acid-B (GABAB) receptor agonists, such as baclofen, GABAA potentiating drugs, such as the benzodiazepines, tumor necrosis factor (TNF)-inhibiting drugs, glucosamine, glucosamine salt, chondroitin, chondroitin salt, hyaluronic acid, Boswellia serrata extract, acetyl-11-keto-boswellic acid, methylsulfonylmethane, collagen type II, L-ergothionine, resveratrol, and other drugs and/or nutritional supplements.

Some embodiments of the present disclosure relate to formulations and therapeutic administration of the formulations with one or more active ingredients that are commonly prescribed or used for treatment of and/or prophylaxis of a degenerative disease/disorder in a subject include, but are not limited to, NSAIDs, COX-2 inhibition, GABAB receptor agonists, such as baclofen, and GABAA potentiating drugs, such as the benzodiazepines.

Some embodiments of the present disclosure relate to formulations and therapeutic administration of the formulations with one or more active ingredients that are commonly prescribed or used for treatment of and/or prophylaxis of a soft tissue disease/disorder in a subject include, but are not limited to, milnacipram, pregabalin, SNRIs, NSRIs, muscle relaxers, sedatives, painkillers, and NSAIDs.

In some embodiments, the novel formulations of the present disclosure may be included in a kit with a separate dosage form containing at least one other active ingredient, exemplified by one or more compounds suitable for the treatment of or commonly prescribed or used for the treating and/or prophylaxis of a joint or inflammatory disease/disorder (e.g. arthritis).

In some embodiments, the novel formulations of the present disclosure may be included in a kit with a separate diagnostic agent or tool exemplified by one or more agents/tools suitable for use as part of a diagnostic test. In certain embodiments the diagnostic agent or tool is used as part of a test for measuring the levels of one or more biomarkers.

### EXAMPLES

Pharmacokinetics (PK) of a cannabis-containing oil formulation and a cannabis-containing gel formulation were investigated. The cannabis was derived from an industrial hemp. The cannabis-containing oil formulation was a full spectrum oil extracted from industrial hemp. In one embodiment, the cannabis-containing oil formulation provides approximately 2.9 wt % total cannabinoids including approximately 2.86 wt % CBD (approximately 26.77 mg/mL) and 0.12 wt % THC (approximately 1.13 mg/mL), providing a CBD:THC ratio of approximately 23.7:1.

The cannabis-containing gel formulation comprises the cannabis-containing oil formulation plus additional excipients. Suitable excipients include, without intending any limitation, protein sources, glucan sources, probiotics, palatability enhancers, and the like. In an embodiment, the cannabis-containing gel formulation is provided as a formulation made by combining the industrial hemp oil described above in an amount to provide 415 mg (46.11%), a yeast cell wall source (12.22%; MacroGard®, Biorigin, Louisville, KY), hydrolized brewer's yeast (35.5%; Biotaste Pet®, Biorigin, Louisville, KY), a palatability enhancer (4.44%; Bioflavor®, Applied Food Biotechnology, Inc., St. Charles, MO), and silicon dioxide (1.72%). The composition was mixed for 15 minutes. The liquid-to-solid phase composition of the final cannabis-containing gel formulation was 46.11% to 53.89%, respectively.

The experiments were performed as a single dose randomized cross-over study wherein eight (n=8) cats were randomized to two treatment groups and crossed-over after a one-week washout. The only effects considered adverse that were seen during the course of the study were 4 incidences of mild vomiting. All animals recovered without intervention. No other adverse effects were observed.

Plasma samples were collected at the following time points: 1) pre-treatment; 2) 1 hour; 3) 2 hour; 4) 4 hour; 5) 8 hour; and 6) 24 hours. At each of those time points, blood samples were collected, spun down to separate plasma, stored, and analyzed for plasma CBD and THC levels. Plasma samples were extracted [Kraemer et al. 2019. Detectability of various cannabinoids in plasma samples of cannabis users: Indicators of recent cannabis use? Drug Test Anal. 11 (10): 1-9] and the extracts were analyzed for CBD and THC by liquid chromatography with tandem mass spectrometry (LC-MS/MS).

Data were subject to PK evaluation using PK Solutions 2.0 (Summit Research Services, 1999). PK parameters (Cmax, Tmax, AUC, and MRT) were subject to logarithmic transformation to adjust for normality and analyzed using one-way ANOVA to compare treatments. One cat was removed for statistical analysis as an outlier.

### Example 1. Plasma CBD Response for Orally Administered Gel Formulation Vs Oil Formulation

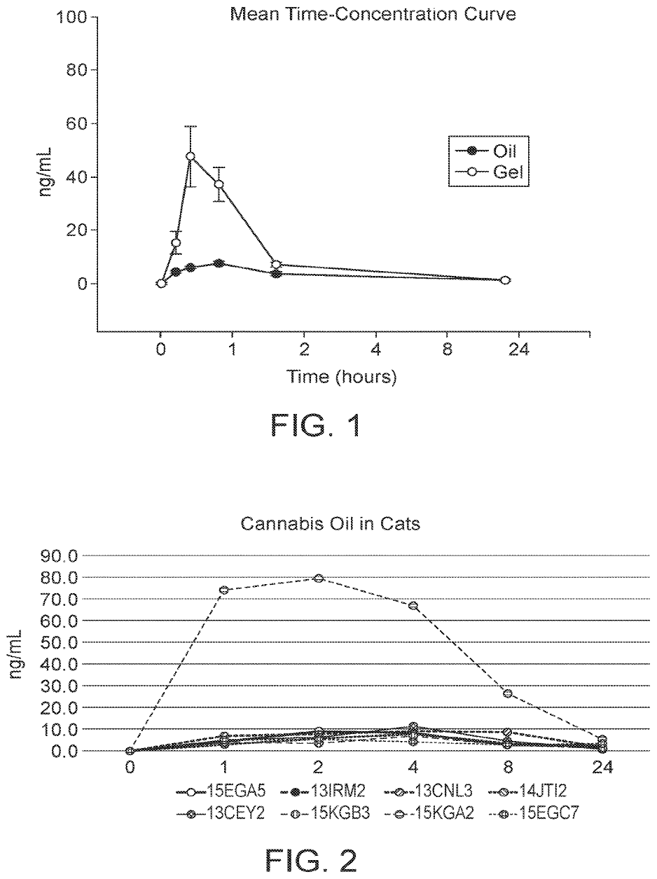

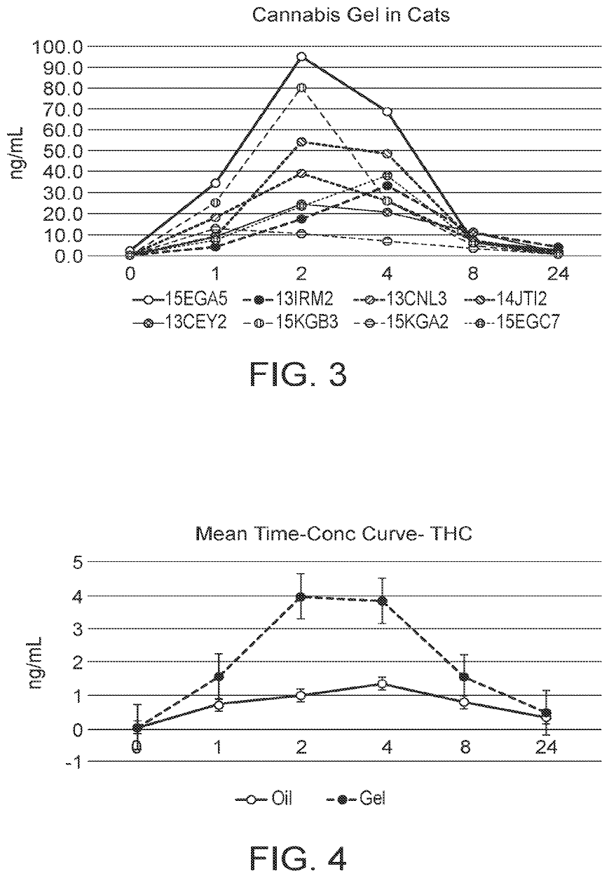

It was surprisingly found that the cannabis composition provided in an orally administered gel formulation significantly increased plasma CBD maximum concentration over time (Cmax), Area under the Curve (AUC; a measure of actual body exposure to the composition after administration of a dose), and mean residence time (MRT) of CBD compared to the cannabis composition provided in an orally administered oil formulation. Indeed, CBD mean peak concentrations (see FIG. 1) were 6.3 times higher for the cannabis-containing gel formulation compared to the oil formulation, translating to a relative bioavailability (F)=1.57. See Tables 1~4 and FIGS. 1-3.

TABLE 1
Plasma CBD Cmax*

| | Mean ± St Dev |
|---|---|
| Oil | 8.20 ± 1.82 |
| Gel | 52.13 ± 26.13 |

Analysis revealed significant differences between the oil and gel (p < 0.001).
*1 cat was removed for statistical analysis as an outlier.

TABLE 2
Plasma CBD AUC*

| | Mean ± St Dev |
|---|---|
| Oil | 89.92 ± 10.11 |
| Gel | 285.7 ± 33.01 |

The treatment groups are significantly different (p < 0.001).
*1 cat was removed for statistical analysis as an outlier.

TABLE 3
Plasma CBD MRT*

| | Mean ± St Dev |
|---|---|
| Oil | .90 ± 37.42 |
| Gel | 7.40 ± 2.93 |

The treatment groups are significantly different (p = 0.005).
*1 cat was removed for statistical analysis as an outlier.

CBD Tmax (time after administration when maximum plasma concentrations are reached) was not significantly different for gel formulations compared to oil formulations. See Table 5.

TABLE 5
Plasma CBD Tmax*

| | Mean ± St Dev |
|---|---|
| Oil | 3.43 ± 0.98 |
| Gel | 2.57 ± 0.98 |

The treatment groups were not statistically different.
*1 cat was removed for statistical analysis as an outlier.

### Example 2. Plasma THC Response for Orally Administered Gel Formulation Vs Oil Formulation

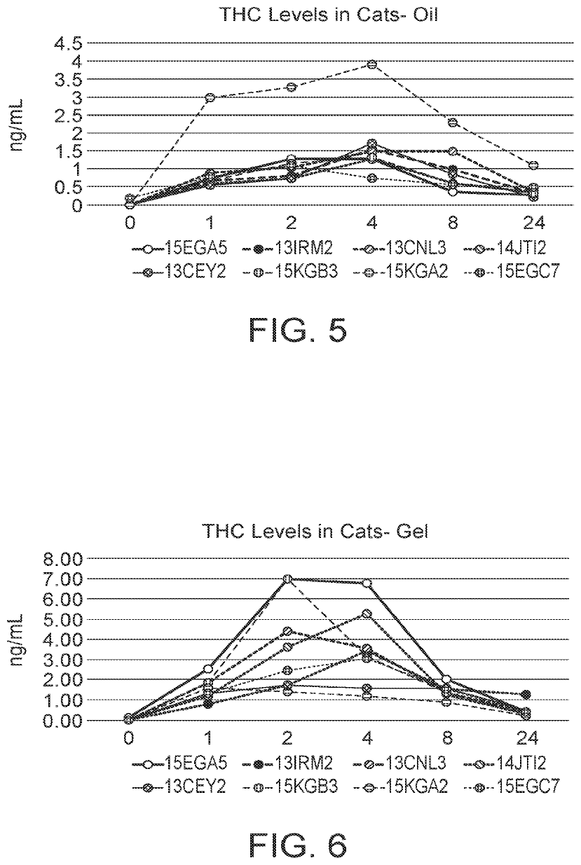

Likewise, oral administration of the cannabis gel formulation significantly increased THC Cmax. See Table 6 and FIGS. 4-6. Oral administration of the cannabis-containing gel formulation significantly decreased THC mean AUC and MRT. See Table 6 and FIG. 4.

TABLE 6
Plasma THC Cmax*

| | Mean ± St Dev |
|---|---|
| Oil | 1.44 ± 0.21 |
| Gel | 4.53 ± 2.00 |

Analysis revealed significant differences between the oil and gel (p < 0.001).
*1 cat was removed for statistical analysis as an outlier.

TABLE 7
Plasma THC AUC*

| | Mean ± St Dev |
|---|---|
| Oil | 16.46 ± 4.07 |
| Gel | 9.18 ± 9.18 |

The treatment groups are significantly different (p < 0.001).
*1 cat was removed for statistical analysis as an outlier.

TABLE 8
Plasma THC MRT*

| | Mean ± St Dev |
|---|---|
| Oil | 35.06 ± 13.41 |
| Gel | 25.49 ± 25.49 |

The treatment groups are significantly different (p = 0.026).
*1 cat was removed for statistical analysis as an outlier.

THC Tmax (time after administration when maximum plasma concentrations are reached) was not significantly different for gel formulations compared to oil formulations. See Table 9.

TABLE 9
Plasma THC Tmax*

| | Mean ± St Dev |
|---|---|
| Oil | 3.43 ± 0.98 |
| Gel | 2.86 ± 1.07 |

The treatment groups were not statistically different.
*1 cat was removed for statistical analysis as an outlier.

As demonstrated by the foregoing studies, bioavailability of cannabinoids was significantly improved by providing in the disclosed gel formulation. While not wishing to be bound by any particular theory, potentially the provision of cannabis-containing gel formulations improved absorption in orally administered dosage forms resulting in the observed improved bioavailability of cannabis administered via a gel formulation compared to a full spectrum hemp oil formulation.

The foregoing descriptions of various embodiments of the disclosure are provided for purposes of illustration and are not intended to be exhaustive or limiting. Modifications or variations are possible in light of the above teachings. The embodiments described above were chosen to provide the best application to thereby enable one of ordinary skill in the art to utilize the disclosed disclosures in various embodiments and with various modifications as are suited to the particular use contemplated. All such modifications and variations are within the scope of the disclosure and appended claims when interpreted in accordance with the breadth to which they are fairly, legally and equitably entitled. All patent and non-patent publications cited herein are incorporated by reference in their entirety.

1. A cannabis-containing composition comprising:
at least one cannabinoid, provided in a gel formulation, the at least one cannabinoid includes Δ⁹-tetrahydrocannabinol (THC) and cannabidiol (CBD),
terpenes, and
wherein the composition is formulated in unit dosage forms suitable for one or more of oral, rectal, intravenous, subcutaneous, intramuscular, and transmucosal administration.

2. The composition of claim 1, wherein a source of the at least one cannabinoid is a hemp oil extract.

3. The composition of claim 1 including at least one of a yeast cell wall source and hydrolized brewer's yeast.

4. The composition of claim 1, wherein the cannabidiol is synthetic.

5. The composition of claim 1 including flavonoids.

6. The composition of claim 1 including excipients and/or carriers selected from the group consisting of vegetable oil, fish oil, krill oil, maltodextrin, calcium carbonate, dicalcium phosphate, tricalcium phosphate, microcrystalline cellulose, dextrose, rice flour, magnesium stearate, stearic acid, croscarmellose sodium, sodium starch glycolate, crospovidone, sucrose, vegetable gums, lactose, methylcellulose, povidone, carboxymethylcellulose, corn starch, and mixtures thereof.

7. The composition of claim 1 including a palatability enhancer and silicon dioxide.

8. A method of treating and/or reducing symptoms of an inflammatory response and/or inflammation, comprising administering to a subject in need thereof a cannabis-containing composition comprising at least one cannabinoid, the composition being provided in a gel formulation, the at least one cannabinoid includes Δ⁹-tetrahydrocannabinol (THC) and cannabidiol (CBD), and wherein the composition is formulated in unit dosage forms suitable for one or more of oral, rectal, intravenous, subcutaneous, intramuscular, and transmucosal administration.

9. The method of claim 8, wherein the composition includes at least one of a yeast cell wall source and hydrolized brewer's yeast.

10. The method of claim 8, wherein a source of the at least one cannabinoid is a hemp oil extract.

11. The method of claim 8, wherein a source of the at least one cannabinoid is cannabis.

12. The method of claim 8, wherein a source of the at least one cannabinoid is a cannabis extract.

13. The method of claim 8, wherein the cannabidiol is synthetic.

14. The method of claim 8, wherein the composition includes terpenes.

15. The method of claim 8, wherein the composition includes flavonoids.

16. The method of claim 8, wherein the composition is formulated for oral administration to an avian and/or a mammalian subject.

17. The method of claim 8, wherein the composition includes excipients and/or carriers selected from the group consisting of vegetable oil, fish oil, krill oil, maltodextrin, calcium carbonate, dicalcium phosphate, tricalcium phosphate, microcrystalline cellulose, dextrose, rice flour, magnesium stearate, stearic acid, croscarmellose sodium, sodium starch glycolate, crospovidone, sucrose, vegetable gums, lactose, methylcellulose, povidone, carboxymethylcellulose, corn starch, and mixtures thereof.

18. The method of claim 8, wherein the composition includes one or more additional active ingredients selected from the group consisting of analgesics, non-steroidal anti-inflammatory drug compounds (NSAID), disease-modifying antirheumatic drugs (DMARDs), corticosteroids, anakinra (an interleukin-1 receptor antagonist), COX-2 inhibition, gamma-aminobutyric acid-B (GABAB) receptor agonists, such as baclofen, GABAA potentiating drugs, such as the benzodiazepines, tumor necrosis factor (TNF)-inhibiting drugs, glucosamine, glucosamine salt, chondroitin, chondroitin salt, hyaluronic acid, Boswellia serrata extract, acetyl-11-keto-boswellic acid, methylsulfonylmethane, collagen type II, L-ergothionine, resveratrol, nutritional supplements, and combinations thereof.

19. A cannabis-containing composition comprising:
hemp oil in an amount of 46.11 weight percent (wt. %),
a yeast cell wall source in an amount of 12.22 wt. %,
hydrolized brewer's yeast in an amount of 35.5%,
a palatability enhancer in an amount of 4.44 wt. %, and
silicon dioxide in an amount of 1.72 wt. %, based on the total weight of the composition.

20. The composition of claim 19, wherein the composition has a liquid-to-solid phase composition of 46.11% to 53.89%.
